(19) United States
(12) Patent Application Publication
(10) Pub. No.: US 2016/0022936 A1
(43) Pub. Date: Jan. 28, 2016

(54) A GAS DELIVERY SYSTEM INCLUDING A FLOW GENERATOR EMPLOYING A CONTINUOUSLY VARIABLE TRANSMISSION

(71) Applicant: KONINKLIJKE PHILIPS N.V., Eindhoven (NL)

(72) Inventor: Thomas V. NIERI, JR., Harrison City, PA (US)

(21) Appl. No.: 14/775,800

(22) PCT Filed: Mar. 12, 2014
(86) PCT No.: PCT/IB2014/059649

§ 371 Date: Sep. 14, 2015

Related U.S. Application Data
(60) Provisional application No. 61/803,845, filed on Mar. 21, 2013.

(51) Int. Cl. A61M 16/00; A61M 16/08

(52) U.S. Cl. CPC A61M 16/0066; A61M 16/0883; A61M 2205/103; A61M 2205/42

(57) ABSTRACT

A gas delivery system for delivering a flow of breathing gas to a patient includes a blower assembly for generating the flow of breathing gas, the blower assembly including a source of rotational energy, such as an electric motor, a continuously variable transmission coupled to the source of rotational energy, and an impeller coupled to the continuously variable transmission.

### BACKGROUND OF THE INVENTION

1. Field of the Invention

The present invention relates to gas delivery systems, and in particular to a gas delivery system having a flow generator that includes a continuously variable transmission.

2. Description of the Related Art

Medical devices that provide a flow of gas to an airway of a patient are used in a variety of situations. For example, ventilators replace or augment a patient's own breathing, pressure support devices deliver pressurized gas to treat breathing disorders, such as obstructive sleep apnea (OSA), and anesthesia machines deliver an anesthesia gas to the patient.

These devices include a flow generator for generating the flow of gas that is delivered to the patient. A typical flow generator includes a motor, such as a brushless electric motor, which drives an impeller, which is often referred to in combination as a blower or blower assembly.

During operation, vibrations that are caused by the blower assembly may cause noise to be generated by the gas delivery system in which the flow generator is mounted. Treatment provided by gas delivery systems are often delivered to the patient while the patient, and any bed partners, are sleeping (or attempting to sleep). Consequently, minimizing sound emission from a gas delivery system is of significant concern.

In addition, the motor is typically the most expensive portion of a blower assembly. Any solution that can help reduce the cost of the motor in a gas delivery system would thus be advantageous.

### SUMMARY OF THE INVENTION

Accordingly, it is an object of the present invention to provide a gas delivery system that overcomes the shortcomings of conventional gas delivery systems. This object is achieved according to one embodiment of the present invention by providing a gas delivery system having a flow generator that includes a continuously variable transmission.

In one embodiment, a gas delivery system for delivering a flow of breathing gas to a patient is provided that includes a blower assembly for generating the flow of breathing gas, the blower assembly including a source of rotational energy, such as an electric motor, a continually variable transmission coupled to the source of rotational energy, and an impeller coupled to the continuously variable transmission.

In another embodiment, a method of generating a flow of breathing gas for a gas delivery system is provided that includes steps of generating rotational energy, and using the rotational energy to drive an impeller by transmitting the rotational energy to the impeller through a continuously variable transmission.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a schematic diagram of a pressure support system according to one particular, non-limiting embodiment of the present invention;

FIG. 2 is a block diagram of a blower assembly forming part of the pressure support system of FIG. 1; and

FIGS. 3-5 are schematic diagrams illustrating various exemplary continuously variable transmission embodiments that may be used in the blower assembly of FIG. 2.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

As used herein, the term “gas delivery system” shall mean a device that delivers a flow of gas to the airway of the patient, invasively or non-invasively.

As used herein, the term “continuously variable transmission” (abbreviated CVT) shall mean a transmission that can change steplessly through an infinite number of effective gear ratios between maximum and minimum values. This contrasts with other mechanical transmissions that offer a fixed number of gear ratios. The flexibility of a CVT allows the input shaft to maintain a constant angular velocity over a range of output velocities.

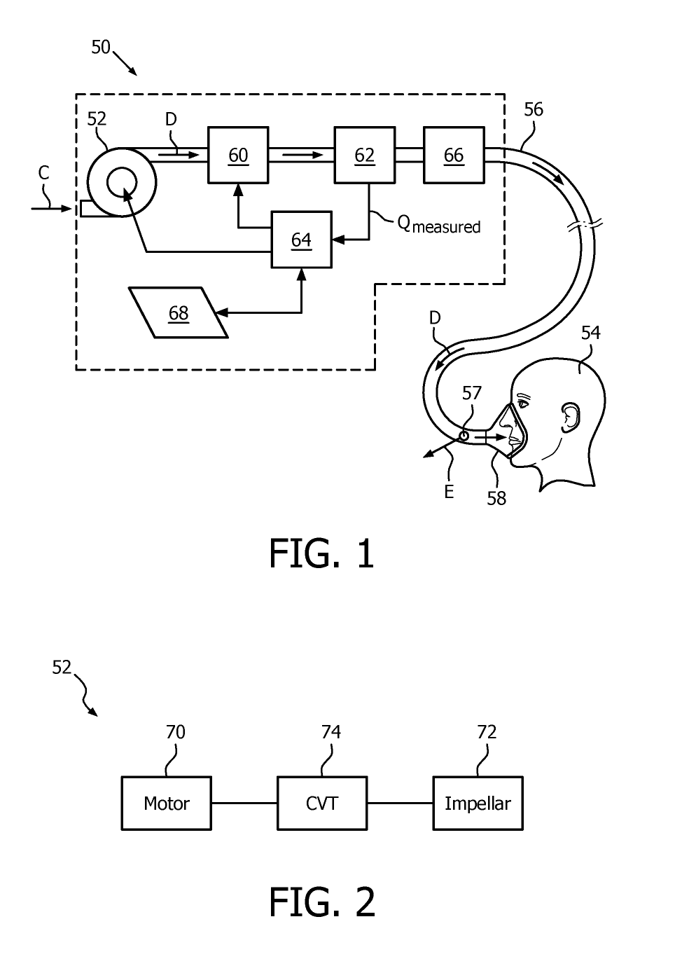

FIG. 1 is a schematic diagram of a pressure support system 50 according to one particular, non-limiting embodiment of the present invention. It should be understood that pressure support system 50 is meant to be exemplary only for purposes of illustrating and describing the present invention, and that the present invention may be implemented and employed in other types of gas delivery systems, such as, without limitation, an invasive or non-invasive ventilator system. One such alternative gas delivery system is described in PCT Publication No. WO 2010/044038, entitled “Volume Control in a Medical Ventilator,” assigned to the assignee of the present invention, the disclosure of which is incorporated herein by reference.

Referring to FIG. 1, pressure support system 50 includes a blower assembly 52 that receives breathing gas, generally indicated by arrow C, from any suitable source, e.g., a pressurized tank of oxygen or air, the ambient atmosphere, or a combination thereof, and generates a flow of breathing gas, such as air, oxygen, or a mixture thereof, for delivery to an airway of a patient 54 at relatively higher and lower pressures, i.e., generally equal to or above ambient atmospheric pressure.

The pressurized flow of breathing gas, generally indicated by arrow D from blower assembly 52, is delivered, via a delivery conduit 56, to a patient interface device 58 of any known construction, which is typically worn by or otherwise attached to patient 54 to communicate the flow of breathing gas to the airway of patient 54. Delivery conduit 56 and patient interface device 58 are typically collectively referred to as a patient circuit.

Although not shown in FIG. 1, the present invention also contemplates providing a secondary flow of gas, either alone or in combination with the primary flow of gas (arrow C) from atmosphere. For example, a flow of oxygen from any suitable source, such as an oxygen concentrator, or oxygen storage device (liquid or gas), can be provided upstream of blower assembly 52 or downstream of blower assembly 52, for example in the patient circuit or at the patient interface device, to control the fraction of inspired oxygen delivered to the patient.

Pressure support system 50 shown in FIG. 1 is a single-limb system, meaning that the patient circuit includes only delivery conduit 56 connecting patient 54 to pressure support system 50. An exhaust vent 57 is provided in the delivery conduit 56 for venting exhaled gasses (e.g., CO₂) from the system to atmosphere as indicated by arrow E. In the exemplary embodiment, the patient circuit is a passive circuit and exhaust vent 57 is a fixed orifice. It should be noted that exhaust vent 57 can be provided at other locations in addition to or instead of in delivery conduit 56, such as in patient interface device 58. It should also be understood that exhaust vent 57 can have a wide variety of configurations depending on the desired manner in which gas is to be vented from pressure support system 50.

In the illustrated exemplary embodiment, patient interface 58 is a nasal/oral mask. It is to be understood, however, that patient interface 58 can include a nasal mask, nasal pillows, tracheal tube, endotracheal tube, or any other device that provides the gas flow communicating function. Also, for purposes of the present invention, the phrase “patient interface” can include delivery conduit 56 and any other structures that connect the source of pressurized breathing gas to patient 54.

In the illustrated embodiment, pressure support system 50 includes a pressure controller or flow controller in the form of a valve 60 provided in delivery conduit 56. Valve 60 controls the pressure or the flow of breathing gas from gas blower assembly 52 delivered to patient 54. For present purposes, blower assembly 52 and valve 60 are collectively referred to as a “pressure generating system” because they act in concert to control the pressure and/or flow of gas delivered to the patient.

It should be apparent that other techniques for controlling the pressure or the flow delivered to patient 54 by blower assembly 52, such as varying the blower speed, either alone or in combination with a pressure control valve, are contemplated by the present invention. Thus, valve 60 is optional depending on the technique used to control the pressure of the flow of breathing gas delivered to patient 54. If valve 60 is eliminated, the pressure generating system corresponds to blower assembly 52 alone, and the pressure of gas in the patient circuit is controlled, for example, by controlling the motor speed of the blower assembly 52.

Pressure support system 50 further includes a flow sensor 62 that measures the flow of the breathing gas within delivery conduit 56. In the particular embodiment shown in FIG. 1, flow sensor 62 is interposed in line with delivery conduit 56, most preferably downstream of valve 60. Flow sensor 62 generates a flow signal, Q_{measured}, which is provided to controller 64 and is used by controller 64 to determine the flow of gas at patient 54 (Qₚₐₜᵢₑₙₜ).

Techniques for calculating Qₚₐₜᵢₑₙₜ based on Q_{measured} are well known, and take into consideration the pressure drop of the patient circuit, known leaks from the system, i.e., the intentional exhausting of gas from the circuit as indicated by arrow E in FIG. 1, and unknown leaks from the system, such a leaks at the mask/patient interface. The present invention contemplates using any known or hereafter developed technique for calculating leak flow Qₗₑₐₖ, and using this determination in calculating Qₚₐₜᵢₑₙₜ based on Q_{measured}. Examples of such techniques are taught by U.S. Pat. Nos. 5,148,802; 5,313,937; 5,433,193; 5,632,269; 5,803,065; 6,029,664; 6,539,940; 6,626,175; and 7,011,091, the contents of each of which are incorporated by reference into the present invention.

Pressure support system 50 also includes a pressure sensor 66 that measures the pressure of the breathing gas within delivery conduit 56. In the particular embodiment shown in FIG. 1, pressure sensor 66 is interposed in line with delivery conduit 56.

Of course, other techniques for measuring the respiratory flow of patient 54 and the pressure of gas delivered to patient 54 are contemplated by the present invention, such as, without limitation, measuring the flow and/or pressure directly at patient 54 or at other locations along delivery conduit 56, measuring patient flow and/or pressure based on the operation of flow generator 52, and measuring patient flow and/or pressure using a sensor upstream of valve 60.

Controller 64 includes a processing portion which may be, for example, a microprocessor, a microcontroller or some other suitable processing device, and a memory portion that may internal to the processing portion or operatively coupled to the processing portion and that provides a storage medium for data and software executable by the processing portion for controlling the operation of pressure support system 50. Input/output device 68 is provided for setting various parameters used by pressure support system 50, as well as for displaying and outputting information and data to a user, such as a clinician or caregiver.

FIG. 2 is a block diagram of blower assembly 52 according to an exemplary embodiment of the present invention. As seen in FIG. 2, blower assembly 52 includes a motor 70 (e.g., a brushless electric motor) which is operatively coupled to an impeller 72 (or multiple impellers) through a continuously variable transmission (CVT) 74. In the exemplary embodiment, motor 70, impeller 72 and CVT 74 are enclosed within a blower housing (not shown) which defines an air inlet and a flow outlet. As is known, impeller 72 includes a plurality of blades such that when impeller 72 is rotatably driven by motor 70 and CVT 74, the blades force air contained within the blower housing to exit the blower housing through the flow outlet. As the air in the blower housing is forced out of the flow outlet, air is drawn into the blower housing through the air inlet.

Thus, as just described, blower assembly 52 includes integration of CVT 74 between electric motor 70 (or an alternative suitable source of rotational energy, such as, without limitation, a hydraulic motor, a pneumatic motor, a combustion engine, a steam turbine, a gas turbine, or a manual crank) and impeller 72 as a transmission mechanism for transmitting the rotational energy of motor 70 to impeller 72. Integration of CVT 74 in this manner would allow the manufacturer of pressure support system 50 to select a lower cost electric motor for motor 70 to provide therapy, as the torque and power needed to generate the required high therapy pressures, blower speeds, and quick speed/pressure changes could be achieved through the mechanical advantage created by CVT 74 as opposed to the design of motor 70 alone.

In addition, integration of CVT 74 would also allow motor 70 to run at lower speeds (than without CVT 74), which would in turn create less electromechanical noise and vibration. Furthermore, since the blower speed/pressure would be mostly changed by adjusting CVT 74, any electromechanical resonance associated with changing the speed of motor 70 to change pressure would be mitigated/eliminated.

CVT 74 may take on any of a number of different structures and/or configurations, including, without limitation, any of the following known CVT configurations: a variable diameter pulley (VDP) or Reeves drive, a toroidal CVT, a magnetic CVT, an infinitely variable transmission, a ratcheting CVT, a hydrostatic CVT, a cone CVT, a radial roller CVT, a planetary CVT.

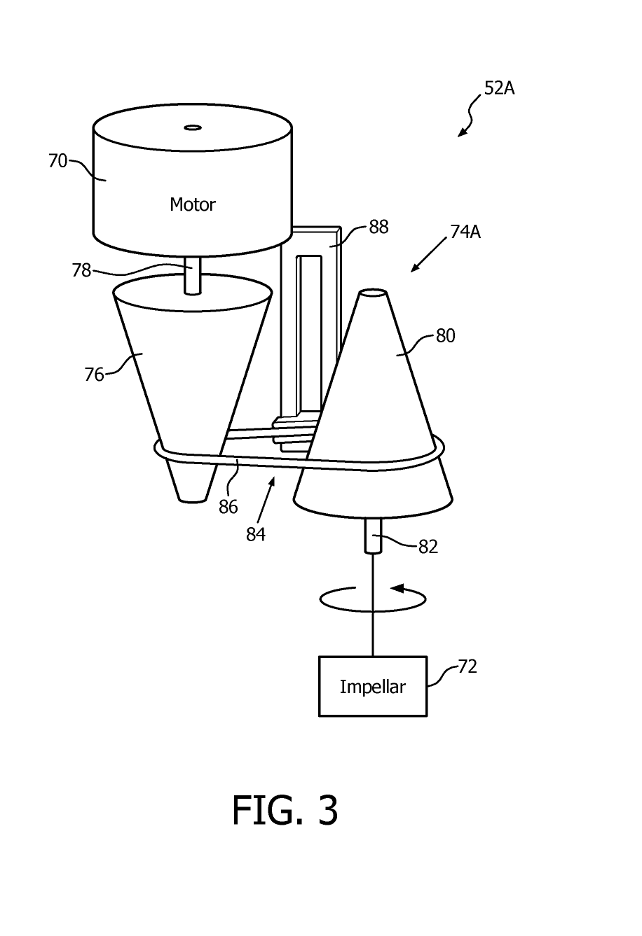

FIG. 3 is a schematic diagram of a particular, non-limiting exemplary embodiment of blower assembly 52, labeled 52A, which may be employed in pressure support system 50. Blower assembly 52A employs a cone type CVT, labeled 74A. As seen in FIG. 3, CVT 74A includes a motor cone 76 coupled to motor 70 through a shaft 78, and an impeller cone 80 coupled to impeller 72 through a shaft 82. A belt system 84 couples motor cone 76 to impeller cone 80. Belt system 84 includes a belt member 86 coupled to and extending between motor cone 76 and impeller cone 80, and a linear actuator 88 coupled to belt member 86 and structured to adjust the position of belt member 86 on motor cone 76 and impeller cone 80 (e.g., linear actuator 88 may be driven by a servo motor). In operation, rotational motion from motor 70 spins motor cone 76, and this rotational motion would be transferred to impeller cone 80 by belt system 84. The infinite gear ratio between motor cone 76 and impeller cone 80 is able to be changed by using linear actuator 88 to vertically position belt member 86 to achieve the desired blower speed (i.e., rotational speed of impeller 72).

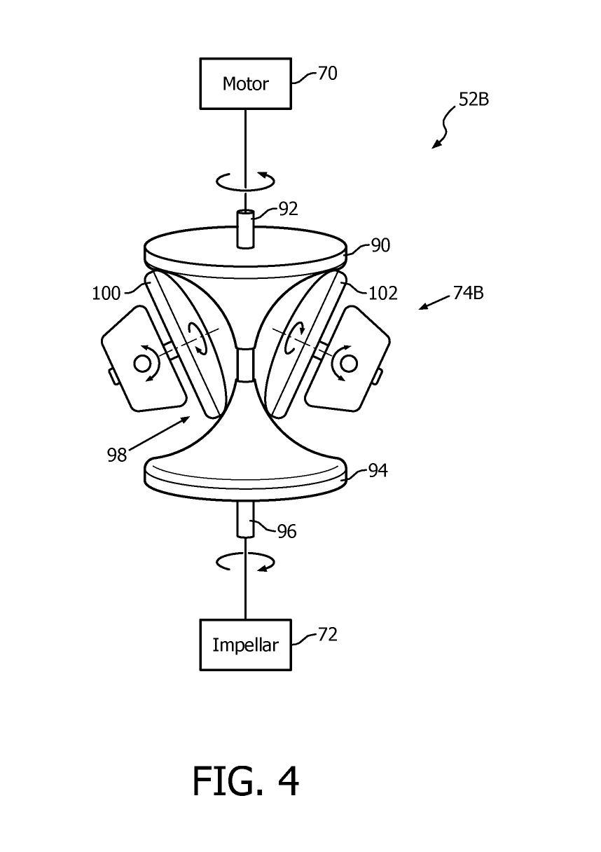

FIG. 4 is a schematic diagram of another particular, non-limiting exemplary embodiment of blower assembly 52, labeled 52B, which may be employed in pressure support system 50. Blower assembly 52B employs a toroidal CVT, labeled 74B. As seen in FIG. 4, CVT 74B includes an input disk 90 coupled to motor 70 through a shaft 92, and an output disk 94 coupled to impeller 72 through a shaft 96. The input disk 90 and the output disk 94 can be pictured as two almost conical parts, point to point, with the sides dished such that the two parts could fill the central hole of a torus. A roller mechanism 98 including first rotatably adjustable roller 100 and second rotatably adjustable roller 102 couples input disk 90 to output disk 94 and transmits rotational motion between input disk 90 to output disk 94 according to a ration that may be varied depending on the position of roller mechanism 98. The position of roller mechanism 98 is controlled by the use of an electric servo motor. In particular, when the axis of roller mechanism 98 is perpendicular to the axis of input disk 90 and output disk 94, it contacts the parts at same-diameter locations and thus gives a 1:1 gear ratio. The roller mechanism 98 can be moved along the axis of input disk 90 and output disk 94, changing angle as needed to maintain contact. This will cause roller mechanism 98 to contact input disk 90 and output disk 94 at varying and distinct diameters, giving a gear ratio of something other than 1:1.

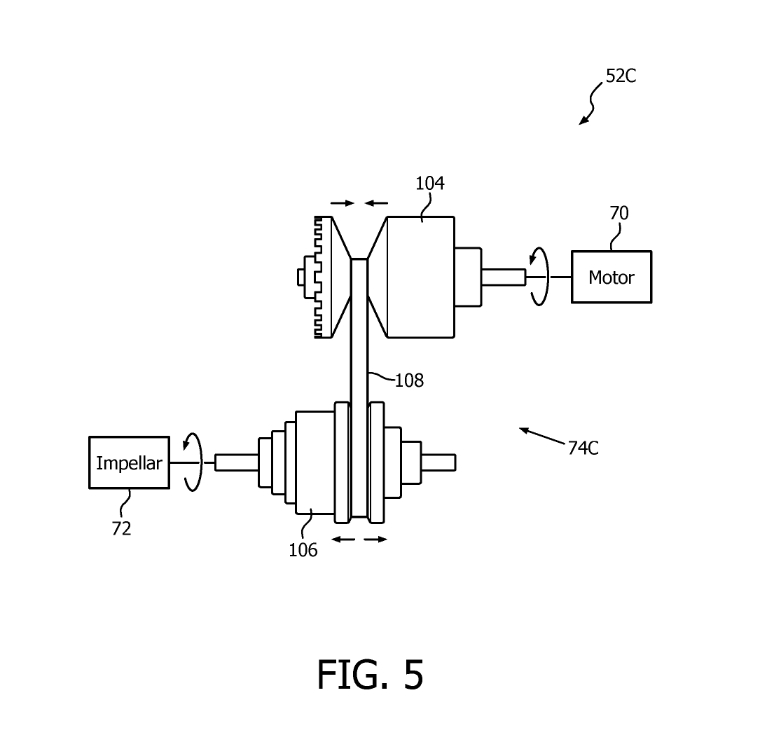

FIG. 5 is a schematic diagram of yet another particular, non-limiting exemplary embodiment of blower assembly 52, labeled 52C, which may be employed in pressure support system 50. Blower assembly 52C employs a variable diameter pulley CVT, labeled 74C. As seen in FIG. 4, CVT 74C includes a first pulley mechanism 104, a second pulley mechanism 106, and a V-shaped drive belt 108 proved between and coupled to first pulley mechanism 104 and second pulley mechanism 106. First pulley mechanism 104 is coupled to and driven by motor, and second pulley mechanism 106 is coupled to and drives impeller 72. First pulley mechanism 104 and second pulley mechanism 106 each contain two cone-shaped sides (split perpendicular to their axes of rotation) on which drive belt 108 would track. The distance between each pulley side of first pulley mechanism 104 and second pulley mechanism 106 is be adjustable (see the arrows in FIG. 5), so that the ratio between first pulley mechanism 104 and second pulley mechanism 106 may be changed. In one non-limiting exemplary embodiment, the distance between each pulley side on either the drive side or the driven side is mechanically controlled, while the non-mechanically controlled side is spring loaded. This would allow the non controlled pulley mechanism to maintain constant belt tension as the controlled pulley mechanism's width is changed. Since the distance between the two pulley mechanisms and the length of drive belt 108 do not change, changing the gear ratio means both pulley mechanisms must be adjusted (one bigger, the other smaller) simultaneously in order to maintain the proper amount of tension on the belt.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A gas delivery system for delivering a flow of breathing gas to a patient, comprising:
a blower assembly for generating the flow of breathing gas, the blower assembly including:
a source of rotational energy;
a continuously variable transmission coupled to the source of rotational energy; and
an impeller coupled to the continuously variable transmission; and
a patient circuit having a patient interface device coupled to the blower assembly for delivering the flow of breathing gas to an airway of the patient.

2. The gas delivery system according to claim 1, wherein the source of rotational energy is a motor.

3. The gas delivery system according to claim 2, wherein the motor is an electric motor.

4. The gas delivery system according to claim 3, wherein the motor is a brushless electric motor.

5. The gas delivery system according to claim 1, wherein the continuously variable transmission is a cone type CVT.

6. The gas delivery system according to claim 1, wherein the continuously variable transmission is a toroidal CVT.

7. The gas delivery system according to claim 1, wherein the continuously variable transmission is a variable diameter pulley CVT.

8. The gas delivery system according to claim 1, wherein the continuously variable transmission is selected from a group consisting of a variable diameter pulley or Reeves drive, a toroidal CVT, a magnetic CVT, an infinitely variable transmission, a ratcheting CVT, a hydrostatic CVT, a cone CVT, a radial roller CVT, and a planetary CVT.

9. The gas delivery system according to claim 1, wherein the source of rotational energy is selected from a group consisting of an electric motor, a hydraulic motor, a pneumatic motor, a combustion engine, a steam turbine, a gas turbine, or a manual crank.

10. A method of generating a flow of breathing gas for a gas delivery system, comprising:
generating rotational energy, and
using the rotational energy to drive an impeller to generate the flow of breathing gas by transmitting the rotational energy to the impeller through a continuously variable transmission;
providing the flow of breathing gas to a patient circuit having a patient interface device for delivering the flow of breathing gas to an airway of the patient.

11. The method according to claim 10, wherein the continuously variable transmission is a cone type CVT.

12. The method according to claim 10, wherein the continuously variable transmission is a toroidal CVT.

13. The method according to claim 10, wherein the continuously variable transmission is a variable diameter pulley CVT.

14. The method according to claim 10, wherein the continuously variable transmission is selected from a group consisting of a variable diameter pulley or Reeves drive, a toroidal CVT, a magnetic CVT, an infinitely variable transmission, a ratcheting CVT, a hydrostatic CVT, a cone CVT, a radial roller CVT, and a planetary CVT.

15-23. (canceled)
